(19) United States
(12) Patent Application Publication
(10) Pub. No.: US 2008/0089854 A1
(43) Pub. Date: Apr. 17, 2008

(54) Skin care compositions

(76) Inventor: Christopher J. Whewell, Georgetown, TX (US)

Correspondence Address: Christopher J. Whewell;Western Patent Group, 6020 Tonkowa Trail, Georgetown, TX, 78628, US

(21) Appl. No.: 12/001,942

(22) Filed: Dec. 13, 2007

Related U.S. Application Data
(63) Continuation-in-part of application No. 11/981,402, filed on Oct. 31, 2007.
Application No. 11/981,402 is a continuation-in-part of application No. 11/978,338, filed on Oct. 29, 2007.
Application No. 11/978,338 is a continuation-in-part of application No. 11/524,556, filed on Sep. 21, 2006, now Pat. No. 7,300,957.

(51) Int. Cl. A61K 8/37; A61Q 1/04; A61Q 15/00; A61Q 19/00; A61Q 5/00

(52) U.S. Cl. 424/64; 424/65; 424/70.1; 514/547

(57) ABSTRACT

Provided are compositions which are suitable for topical application to human skin for cleansing and anti-microbial properties. In one preferred embodiment, the compositions include a glyceryl monoazelate ester. In another embodiment, the compositions include a glyceryl monoazelate monolaurate ester. It is postulated that bacteria on the skin cleave the ester linkage of a compound according to the disclosure that is applied to the skin, which causes liberation of the acid moiety from the ester, which acid moiety then inhibits or kills the bacterium. Compositions according to the disclosure may comprise skin creams, soaps, shampoos and lotions, and are especially effective in treating acne and acne-like skin ebullitions. The glyceryl portion of the molecules is believed to facilitate penetration of the ester into the skin.

### CROSS-REFERENCES TO RELATED APPLICATIONS

This application claims the benefit of U.S. Provisional Application Ser. No. 60/814,147 filed Jun. 16, 2006; U.S. non-provisional application Ser. No. 11/524,556 filed Sep. 21, 2006, now U.S. Pat. No. 7,300,957; U.S. non-provisional application Ser. No. 11/978,338 filed Oct. 29, 2007, currently pending; and U.S. non-provisional application Ser. No. 11/981,402 filed Oct. 31, 2007, currently pending, the entire contents of all of which are herein fully incorporated by reference.

### TECHNICAL FIELD

This disclosure relates generally to compositions of matter which can be applied to mammalian skin and hair, including that of humans, bovine, equestrian, canines, felines, primates and other mammals. More particularly, it relates to personal care compositions useful in managing the appearance and feel of hair and skin.

### BACKGROUND

Various compositions of matter are known to be usefully applied to human skin for a variety of purposes, including emollients, moisturizers, burn treatments, anti-acne, anti-wrinkle, anti-bacterial, anti-carbuncle, pediculicide, etc. In many cases, such compositions include one or more esters as part of their formulations, including di-esters recognized as GRAS under 21 C.F.R. § 1.84. Often, so-called skin creams, vanishing creams, and the like comprise emulsions, in which one or more active ingredients are present in any amount between about 0.001% by weight to about 50% by weight or more, as in the case of concentrates from which emulsions may be prepared. Workers in the prior art have provided a large number of stable skin cream emulsions, many of which are described in expired patents, or patents which are in-force, but not claimed therein. Such prior art patents include, without limitation U.S. Pat. Nos. 6,989,195; 6,903,134; 6,638,621; 6,599,513; 6,596,287; 6,582,710; 6,573,299; 6,552,050; 6,531,117; 6,492,326; 6,464,992; 6,444,647; 6,428,779; 6,403,619; 6,372,234; 6,337,065; 6,284,802; 6,261,575; 6,180,133; 5,876,737; 5,821,237; and 5,126,327 all of which are herein incorporated fully by reference thereto. The production of such topical preparations takes place in the usual way by the active ingredients with suitable additives being converted into the desired application form such as, for example, a solution, a milk, a lotion, a cream, an ointment or a paste. In a preparation thus formulated the concentration of active ingredient depends on the form of application and intended use. It is often preferable that a concentration of 0.1 to 30% by weight, based on the total weight of the final formulation, of active ingredient is used. The milk, lotion or cream (oil/water emulsions) and the ointment (water/oil emulsions) are typically produced in the standard way by use of standard emulsifiers (Kirk Othmer: Encyclopedia of Chemical Technology, 3rd edition, 1979; John Wiley & Sons, New York, Vol 8, pages 900-930, and Dr. Otto-Albrecht Neumueller: Roempps Chemie Lexikon, 7th edition, 1973; Franckh'sche Verlagshandlung Stuttgart, pages 1009-1013), all of which are herein incorporated by reference thereto in their entirety. The waxes, emulsifier, and other additives are the same as conventionally employed used (Dr. Otto-Albrecht Neumueller: Roempps Chemie Lexikon, 7th edition, 1973; Franckhische Verlagshandlung Stuttgart, pages 1427 and 1428), herein incorporated by reference thereto in its entirety.

### SUMMARY OF THE DISCLOSURE

The present disclosure provides compositions of matter useful for topical application to mammalian skin. Compositions according to the disclosure comprise at least one ester compound having a structure selected from the group consisting of: 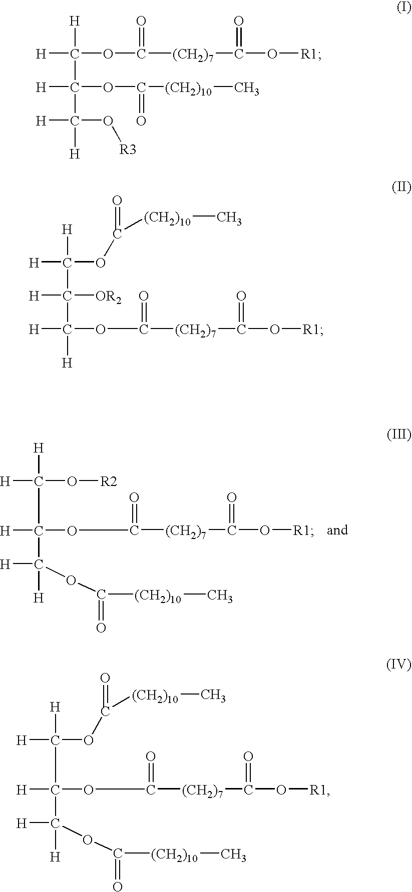
wherein R1, in each occurrence, is selected from the group consisting of: hydrogen, a metal cation, a substituted or unsubstituted ammonium ion, any C1-C18 hydrocarbyl group, and a glyceryl group having the structure: 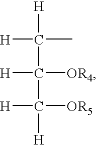
in which R4 and R5 are in each occurrence independently selected from the group consisting of: hydrogen and any C1-C18 hydrocarbyl group; and R2 and R3 are each, in each occurrence, independently selected from the group consisting of: hydrogen, any C1-C18 hydrocarbyl group, and a glyceryl group having the structure: 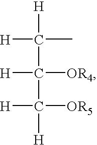
in which R4 and R5 are in each occurrence independently selected from the group consisting of: hydrogen and any C1-C18 hydrocarbyl group. The disclosure also provides methods for applying compositions comprising one or more of the above esters to the skin of a mammal.

### DETAILED DESCRIPTION

The present disclosure concerns the incorporation of one or more novel esters as described herein into milks, lotions, creams, ointments, emulsions and the like which are suitable for application to human hair and skin, wherein the one or more novel esters are present in any amount between about 0.01% to about 90% (including all ranges of percentages therebetween) or more by weight based on the total weight of the finished emulsion, ointment cream, paste, shampoo, concentrate, or other formulation.

The novel ester materials useful as ingredients in a composition according to the present disclosure include without limitation: lauryl phytate; glyceryl phytate; inosityl laureates; lauryl azelate; and glyceryl lauryl azelates (including all positional and stereoisomers thereof), inosityl azelates. Thus, the acids from which esters according to the disclosure may be derived include, without limitation: phytic acid, lauric acid, and azelaic acid, and the alcohols from which esters according to the disclosure may be derived include, without limitation: lauryl alcohol, glycerine, polyalkylene glycols, polyoxyalkylene glycols, and inositol.

There are several methods for producing esters useful according to the present disclosure. In one embodiment, glycerine is directly esterified with the necessary or selected carboxylic acid, using means known in the art of esterification. This includes heating a mixture of glycerine and a base catalyst, present in an effective catalytic amount, to a temperature in the range of between about 80 degrees C. and 180 degrees C, and an appropriate or desired amount of the selected carboxylic acid, one of its salts, such as an alkali metal salt of the carboxylic acid, either the mono- or di-acid salt, as appropriate, depending upon the acid selected. After heating or refluxing with stirring for a few hours, for any amount of time in the range of between about 1 hour to 18 hours, during which liberated condensation product (water, alcohols) is collected in a Dean-Stark trap or side-arm condenser and removed to drive the reaction towards completion. In one embodiment, an excess of glycerine is employed, in order to drive the reaction to completion and to ensure a large relative proportion of mono-ester formation. For example, in the case where lauric acid is added to glycerine under reflux or near boiling in the presence of a base catalyst, a ten-fold excess of glycerine may be used, and the reaction product mixture, after cooling, may be diluted with a large volume of water, into which the un-reacted glycerine is dissolved and separated using a separatory funnel or by decantation from the crude ester and unreacted acid. After rinsing the crude ester several times with an aqueous solution of sodium bicarbonate, traces of residual base catalyst are effectively removed, and the crude ester may be washed with several aliquots of distilled water to yield a clean crude ester product, which may be further worked up and purified by vacuum distillation or molecular distillation. In an alternate embodiment of this method, an ester of the desired carboxylic acid comprising a C1-C18 alcohol substrate may be used in place of the carboxylic acid, including methyl esters like methyl laurate. In such embodiment, methanol, and not water, is liberated as the reaction proceeds.

Another general method of producing an ester useful in accordance with the present disclosure is according to the teachings of Yu et al. in Bull. Korean Chem. Soc. 2003, Vol. 24, No. 8, which is fully incorporated herein by reference thereto. In such embodiment, 1,3-dioxolane-4-methanol, 2,2-dimethyl, a.k.a., the glyceryl ketal of acetone, is employed as a substrate for esterification with a selected acid. In one embodiment, the methyl ester of lauric acid and the glyceryl ketal of acetone are employed as raw materials and the procedure therein followed to yield essentially pure glyceryl monolaurate, itself a powerful antimicrobial agent which is useful as a component in a composition according to the present disclosure and as a raw material from which other esters according to the disclosure may be prepared.

For example, in one embodiment, pure monolaurin (glyceryl monolaurate) as prepared per the foregoing or obtained from Med-Chem Laboratories Inc. of Galena, Ill. (www.lauricidin.com) may be used as a substrate to which an acyl halide is added. One example of an acyl halide so suitable is the acid halide formed from mixing thionyl chloride with methyl hydrogen azelate (Alfa Aesar product L#08116, CAS # 2104-19-0) in a suitable solvent such as toluene, xylene, benzene, etc, which resulting acid halide can be termed methyl azeloyl chloride having structure: 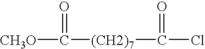
The monolaurin is dissolved in an aprotic solvent such as toluene, and a toluene solution of the above acid-halide is slowly added, to provide a compound having the structure: 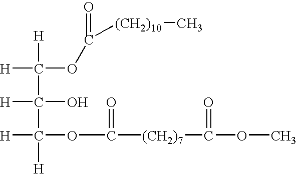
and the solvent may be removed by reduced pressure distillation, leaving a residue which may be purified by conventional means, including without limitation molecular distillation and column chromatography. Although the methyl hydrogen azelate is illustrated above as being a methyl monoester of azelaic acid, other monoesters of azelaic acid may be employed, to provide materials having different R1 groups in the structure (II) above. In analogous fashion, glyceryl monoazelate, i.e., the ester formed from azelaic acid and glycerine, with the azelaic moiety present in either the 1 or 2 position on the glyceryl backbone, may be used as a substrate to which the acid halide lauryl chloride is added, provided that the azelate moiety on the glyceryl ester does not have a free acid group present, but is protected, such as by having an ester thereon. Such materials may be made by directly esterifying glycerine with the monoalkyl ester of the di-acid. Thus, one exemplary substrate material for such addition of lauryl chloride is: 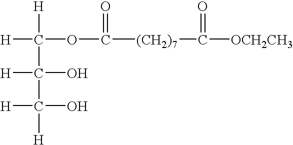
wherein the lauryl chloride may react at either hydroxyl group located at the 2 or 3 position of the glycerine backbone; however, due to steric effects, the predominant product results from the 3-addition:

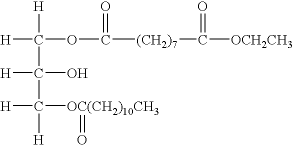

In yet another general method useful for providing a glyceryl lauryl azelate ester useful in accordance with the present disclosure, an glycerine tri-ester oil, such as coconut oil, may be trans-esterified, by heating the oil chosen in the presence of a base (preferably) or acid catalyst and adding the desired carboxylic acid, or its acyl or ester derivative. For example, beef tallow, soybean oil, coconut oil, peanut oil, or any animal-derived oils or plant-derived oils which are predominantly comprised of triesters of glycerine may be heated to a temperature in the range of between about 80 degrees C. and 180 degrees C. in the presence of an effective catalytic amount of acid or base catalyst, to which is added azelaic acid, a metal salt thereof (including without limitation: transition metal salts, alkali metal salts, alkaline earth metal salts) or a monoester thereof.

In yet another embodiment, azelaic acid may be mixed with monolaurin on a 1:1 molar basis, heated to about 135 degrees centigrade in the presence of an effective catalytic amount of a catalyst (including without limitation toluene sulfonic acid, phosphoric acid, tetrapropyl titanate and dibutyltin oxide) and a polymerization inhibitor such as hydroquinone or p-methoxyphenol or other known inhibitors until about one mole of water has been removed, and the reaction product then subject to purification by molecular distillation, column chromatography or other known separation techniques.

The selected carboxylic acid or esters thereof which are suitable for use as raw materials in the foregoing preparative methods for providing an additive for a personal care composition (to be applied to hair or skin) or animal ointment according to the disclosure include without limitation: azelaic acid, mono-alkyl azelaic acid esters derived from any C1 to C18 alcohol (such as monomethyl azelate, monoethyl azelate, monopropyl azelate, etc.); di-alkyl azelaic acid esters derived from any C1 to C18 alcohol, lauric acid (such as dimethyl azelate, dimethyl azelate, dipropyl azelate, etc.), mono-alkyl lauric acid esters derived from any C1 to C18 alcohol (such as methyl laurate, ethyl laurate, propyl laurate, etc.), salts of azelaic acid, and all forms of phytic acid, including any of its various known salts. In one embodiment, azelaic acid and its lower C1 to C6 alkyl esters are especially preferred in providing mixed esters of azelaic acid and lauric acid with glycerine.

An ester useful as a component in a skin-care or hair-care composition according to preferred embodiments of the present disclosure comprises one or more compounds having a structure selected from the group consisting of: 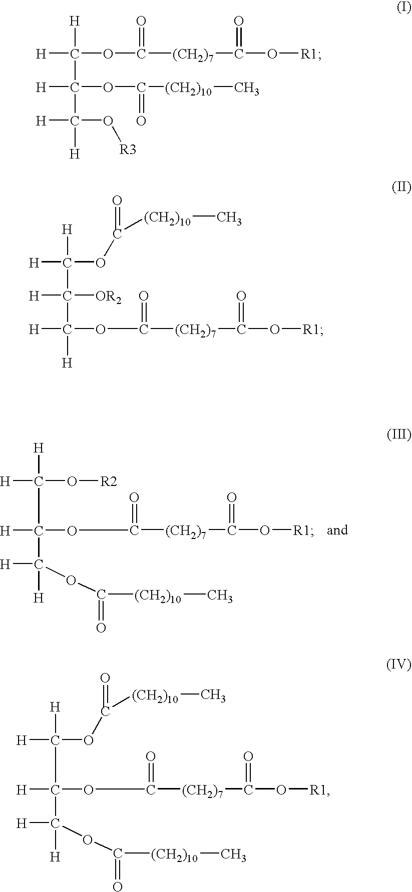
wherein R1, in each occurrence, is selected from the group consisting of: hydrogen, a metal cation, a substituted or unsubstituted ammonium ion, any C1-C18 hydrocarbyl group, and a glyceryl group having the structure: 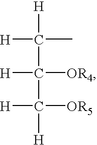
in which R4 and R5 are in each occurrence independently selected from the group consisting of: hydrogen and any C1-C18 hydrocarbyl group; and
R2 and R3 are each, in each occurrence, independently selected from the group consisting of: hydrogen, any C1-C18 hydrocarbyl group, and a glyceryl group having the structure: 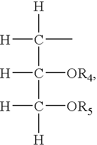
in which R4 and R5 are in each occurrence independently selected from the group consisting of: hydrogen and any C1-C18 hydrocarbyl group.

In the foregoing materials, R1 may be a glyceryl group having the structure: 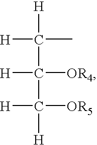
in which R4 and R5 are in each occurrence independently selected from the group consisting of: hydrogen and any C1-C18 hydrocarbyl group, because of some coupling to the free-swinging carboxyl end of an azelate group which is only bound to a glycerine portion of the molecule at one end, which nearly always may occur to some extent during an esterification or other reaction to provide materials specified herein.

The esters of glycerine (“glyceryl lauryl azelates”, or “GLA”) which comprise an azelaic acid and a lauric acid group (structure (I) in which R1, R3=H; structures (II), (III) in which R1, R2 are both hydrogen; and structure (V) in which R1 is H) are excellent emollients, as are those in which R1 are alkyl groups (esters of GLA's). Additionally, these materials make excellent moisturizing creams when present with water in conventional moisturizing cream formulations. They are also environmentally-friendly, being formed from azelaic acid and lauric acid, both of which fatty acids occur in nature. Azelaic acid has the structure: 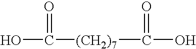
and is found naturally in wheat, rye, and barley, in addition to its being produced by Malassezia furfur (pityrosporum ovale), a yeast that lives on normal skin. Azelaic acid is a naturally-occurring saturated di-carboxlylic acid that is normally found in the human diet and is not considered to be a carcinogenic substance. Mutagenicity studies are negative and animal studies have shown no adverse effects on fertility or reproduction. Human problems have not been reported during pregnancy in association with azelaic acid (http://www.minoxidil.com/azelaic.htm). Thus, a GLA material according to certain embodiments of this disclosure are diglycerides, made from side chains (lauric and azelaic) which are food-derived.

The mixed esters of glyceryl lauryl azelate appear to penetrate the skin well, and the enzyme esterase in the skin is capable of cleaving an azelate group from a GLA following its topical application. Such cleavage leaves not only an azelate species or azelaic acid, but a molecule of monolaurin as well (which itself is a GRAS material specified in 21 C.F.R § 184), which monolaurin itself is also antimicrobial according to U.S. Pat. No. 5,660,842 and the published works of Professor Emeritus Jon Kabara, PhD. (http://www.lauricidin.com). At present, skin creams comprising 20% by weight azelaic acid are employed as acne treatments, but have a high potential for irritating skin. Additionally, only about 3.6% of topically applied azelaic acid is absorbed (Tauber et al., Experimental Dermatology, Volume 1, Issue 4, pp. 176-179, November 1992). The GLA's provided by this disclosure (structures I, II, III) comprise about 40% by weight of azelate groups, and the GLA's provided herein appear to be completely absorbed when applied in neat form, with absolutely no skin irritation whatsoever. This translates to an amount of 55.5 times more azelate potentially being absorbed from application of a GLA as provided herein versus a conventional composition designed to be applied to skin. Thus, a cream comprising just one percent of a GLA is likely to be more than twice as effective as a skin cream which contains 20% azelaic acid formulated for topical application. Accordingly, a composition containing just 5% of a GLA as taught herein may be ten times effective as a composition containing 20% azelaic acid.

The term “hydrocarbyl”, as used in this specification and the claims appended hereto, refers to a hydrocarbon group having a carbon atom directly attached to the remainder of the molecule and having predominantly hydrocarbon character. Examples of hydrocarbyl substituents or groups within this definition include: (1) hydrocarbon substituents, that is, aliphatic (e.g., alkyl or alkenyl, including straight-chain or branched), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form an alicyclic radical); (2) substituted hydrocarbon substituents, that is, substituents containing non-hydrocarbon groups which, in the context of this disclosure, do not alter the predominantly hydrocarbon substituent (e.g., halo (especially chloro and fluoro), hydroxy, carboxy (including C1-C24 carboxylate groups), alkoxy, mercapto, alkylmercapto, nitro, nitroso, and sulfoxy); (3) hetero substituents, that is, substituents which, while having a predominantly hydrocarbon character, in the context of this disclosure, contain other than carbon in a ring or chain otherwise composed of carbon atoms. Heteroatoms include sulfur, oxygen, nitrogen, and encompass substituents such as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in a hydrocarbyl group, with methyl and ethyl groups comprising preferred embodiments of hydrocarbyl groups, and all C1-C24 carboxylic acids being included as hydrocarbyl.

It is known in the art of esterification and transesterification of glycerine that it is rare for the product of such reactions to comprise a single molecule as a reaction product. Rather, mixtures of esters are typically obtained owing to the presence of several often-complex and competing reaction equilibria. Alteration of such variables as reaction time; temperature; reactant and product composition and concentration; pH; presence, nature and identity of catalyst(s) present; and pressure will tend to shift the positions of the various competing equilibria, and typically complex mixtures result even from the addition of lauric acid to heated tallow in the presence of a base or acid, since acid catalysts, including without limitation sulfonic acids and carboxylic acids, are also known to also catalyze esterifications and transesterifications. Thus, the crude mixture resulting from a reaction directed at producing one or more materials described in formula (I) through (IV) above will often comprise a multi-component mixture of glyceryl lauryl azelates. For purposes of this specification and the claims appended hereto, the words “glyceryl lauryl azelate” or “GLA” include any compound described by formulae (I) through (IV) and the text above.

It has been seen that in the foregoing description, the appendage R1 is present on a carboxyl function of the glyceryl lauryl azelate ester. While R1 may represent many hydrocarbyl groups, such as alkyl groups; however, R1 may also comprise a cationic species. Within this context, R1 may comprise any alkali metal, alkaline earth metal, Group III cations (boron, aluminum, et al.) or transition metal cation. Additional suitable cationic species include ammonium cations, mono-alkylammonium cations, di-alkylammonium cations, tri-alkylammonium cations, and quaternaryalkylammonium cations. For alkylated ammonium cations, the alkyl group(s) may comprise any number of carbon atoms from about 1 to about 24. Salts according to this disclosure in which R1 comprises a cationic species may be prepared in the same fashion as metallic carboxylate salts are prepared, such as by reacting the acid form of the carboxylic acid with a basic metal salt, such as an oxide, hydroxide, or carbonate, or alternatively by displacement reactions. In the case of ammonium salts, ammonia or a gaseous amine may be used to treat an aqueous solution of the acid form of the carboxylic acid, the water evaporated and the product recovered. Alternatively, displacement reactions are suitable as well. When the cationic species selected is multivalent, charge balance naturally needs to be maintained and in the case of a tri-positive cation such as aluminum, only one aluminum cation is required to be present for each three anionic species comprising a glyceryl lauryl azelate salt according to the disclosure. This is one especially useful employment to the compounds of the disclosure, since, when the structure of material is that of a glyceryl lauryl azelate ester in any of the formulae above in which R1 is an aluminum or zirconium cation, the compositions of the disclosure are useful as additives in anti-perspirant compositions, owing to their unique property of being soluble in both oils and in aqueous systems. This may in some cases enable deeper penetration into the middle layers of the skin than many other products, to put more GLA at the site of undesirable microbe populations. Exemplary are materials of this formula: 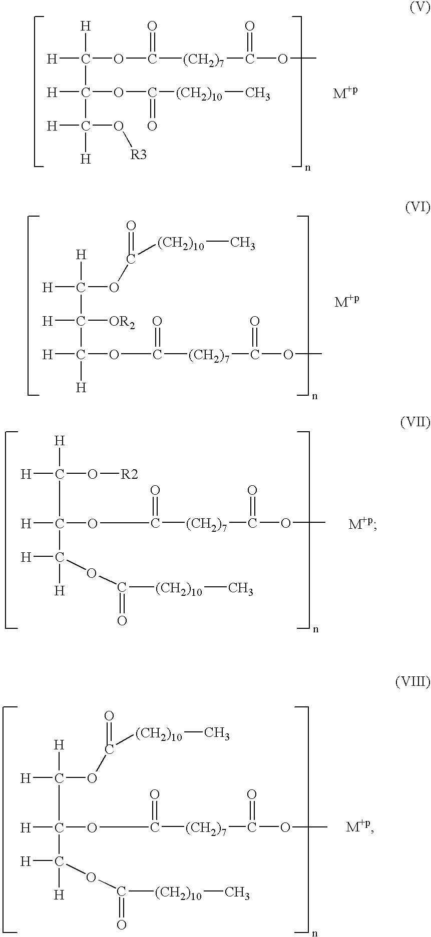
wherein R2, R3 are as defined previously, and in which M may be any alkali metal cation, alkaline earth metal cation, Group III metal cation (boron, aluminum, et al.), any transition metal cation, an ammonium cation, a mono-alkylammonium cation, a di-alkylammonium cation, a tri-alkylammonium cation, and any quaternaryalkylammonium cations, with p being the charge on the cation, and n being the number of GLA anions associated with the species M. By non-limiting example, when M is selected to be zinc, p=2 and n=2; when M is selected to be aluminum, M=3 and p=3; when M is selected to be silver, p=1 and n=1; when M is selected to be an ammonium cation or an alkali metal, M=1 and p=1; and when M is selected to be an alkaline earth cation, M=2 and p=2. These various salts may not be completely dissociated, with the exception of the alkali metal salts. When M is hydrogen, these materials are carboxylic acids with low dissociation constants. When M is monovalent silver, these compounds are excellent as components of burn and wound dressings.

Selection of a desired cation can be made after the hydrolysis step when producing a composition of the disclosure using the glyceryl ketal of acetone and a di-ester of azelaic acid. Alternatively, to illustrate another principle useful in accordance with producing compounds according to the disclosure, monosodium azelate may be esterified with methanol to yield monosodium monomethyl azelate which can be added in powder form to a heated and stirred quantity of the acetone ketal of glycerine, but the yield is likely lower when using this approach versus post neutralization of the acid form of glyceryl lauryl azelate that is made using azelate di-esters as a reactant with the acetone ketal of glycerine. However, the hydrolysis step involving de-protection of the ketal can be performed with the slow addition of a weakly acidic substance comprising the desired cation over time, such as lithium bicarbonate, aluminum bicarbonate, zinc carbonate, copper carbonate, silver oxide, ammonia, diethylamine, alkanolamines, dimethylaminoethanol, alkanolamines, etc. so that the cation is incorporated during the hydrolysis, thus minimizing the propensity for precipitates to form when long alkyl groups are present on R2 and R3.

Alternatively, anionic glyceryl lauryl azelate species may be formed in situ, upon mixing the acid form wherein R1 is hydrogen in formula (I) with the other ingredients of a cream, emulsion, shampoo or other skin care formulation, by simple replacement reactions.

In another alternate embodiment, the method of Synthetic Example 1 of U.S. Pat. No. 4,661,519 (fully incorporated herein by reference thereto) is employed, using a molecular equivalent amount of monolaurin in the stead of specified molar amount of glycerine proscribed therein, to afford a GLA material having structure (II) above in which R1 and R2 are both hydrogen, which may or may not be further purified. Subsequent addition of a slurry of a carbonate or hydroxide salt of the metal cation selected provides the material as in structure (VI) above. Materials according to structure (VI), (VII) in which R2 is hydrogen, and structure (V) in which R3 is hydrogen, all further in which M is zinc, n=2, and p=2 are believed to be effective at restoring hair growth when applied topically to skin having dormant hair follicles.

Generally, topical application to skin sites is accomplished in association with a carrier, and particularly one in which the active ingredient is soluble per se or is effectively solubilized (e.g., as a solution, emulsion or microemulsion). It is necessary that the carrier be inert in the sense of not bringing about a deactivation or de-esterification of the glyceryl lauryl azelate or glyceryl lauryl azelates present in the formulation and the pH of a final composition should be adjusted to about 6.5-7.5.

Suitable carriers include water, alcohols, oils and the like, chosen for their ability to dissolve or disperse the active ingredients at concentrations of active ingredients most suitable for use in the therapeutic treatment. Generally, even low concentrations of GLA materials in a carrier are suitable for their moisturizing and emollient effects. As a practical matter in one embodiment it is desirable that compositions for topical application be formulated to contain at least about 0.25% to about 5% by weight, more preferably from about 1% to about 3% by weight, glyceryl lauryl azelates, salts, esters, or derivatives thereof, and accordingly, carriers will be chosen which can solubilize or disperse these ingredients at such concentrations. In one embodiment, glyceryl lauryl azelate esters are present in a composition according to the disclosure in any amount between about 0.01% to about 80% by weight based on the total weight of the finished emulsion containing the ester. In another embodiment, glyceryl lauryl azelate esters are present in a composition according to the disclosure in any amount between about 0.1% to about 30% by weight based on the total weight of the finished emulsion containing the GLA. The term GLA and the words “glyceryl lauryl azelate” as used herein includes the compounds described by structures (V), (VI), (VII), and (VIII) and the text above, including all salts thereof. One efficacious embodiment for moisturizing skin contains about 2% by weight total glyceryl lauryl azelates content.

While the carrier for glyceryl lauryl azelate can consist of a relatively simple solvent or dispersant such as oils, it is generally preferred that the carrier comprise a composition more conducive to topical application, and particularly one which will form a film or layer on the skin to which it is applied so as to localize the application and provide some resistance to perspiration and/or one which aids in percutaneous delivery and penetration of the active ingredients into lipid layers. Many such compositions are known in the art, and can take the form of lotions, creams, gels or even solid compositions (e.g., stick-form preparations). Typical compositions include lotions containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions may be referred to as dermatologically acceptable carriers. Most preferred for skin are those carriers which are fat-soluble, i.e., those which can effectively penetrate skin layers and deliver the active glyceryl lauryl azelate or glyceryl lauryl azelates to the lipid-rich layers of the skin. In addition, an ester according to the disclosure may be applied using a time-release patch, as are used in hormone delivery, nicotine patches, anti-acne patches, and the like as they are believed useful as adjuvants in enhancing transdermal delivery of drugs. Cremes, aqueous solutions, pastes, powders, etc. are all suitable delivery vehicles for an ester described herein to the human body.

Thus, the glyceryl lauryl azelate esters of the present disclosure may be used in a wide range of personal care compositions (compositions suitable to be applied to either hair or skin or both), as an additive at levels ranging from 1% to 90% by weight based on the total weight of the composition. In addition, the glyceryl lauryl azelate esters of the present disclosure may be blended with other surfactants and materials which are used in personal care products at glyceryl lauryl azelate ester levels ranging up to about 60% by weight. To the extent that other surfactants may be used in combination with the glyceryl lauryl azelate esters of the present disclosure in forming binary active systems, ternary active systems etc., the glyceryl lauryl azelate ester may comprise the majority of an anti-microbial additive system or it may comprise less than the majority of the anti-microbial additive system in which case it is referred to as the secondary additive. Surfactants and materials which may be used in combination with the glyceryl lauryl azelate esters in forming a composition according to the disclosure include without limitation: amphoteric/zwitterionic surfactants; anionic surfactants; nonionic surfactants; cationic surfactants; and optional ingredients, including those described below.

Amphoteric surfactants suitable for inclusion in a personal care composition along with a glyceryl lauryl azelate according to the present disclosure can broadly be described as surface active agents containing at least one anionic and one cationic group and can act as either acids or bases depending on pH. Some of these compounds are aliphatic derivatives of heterocyclic secondary and tertiary amines in which the aliphatic radical may be straight or branched and wherein one of the aliphatic substituents contains from about 6 to about 20, preferably 8 to 18, carbon atoms and at least one contains an anionic water-solubilizing group, e.g., carboxy, phosphonate, phosphate, sulfonate, sulfate.

Zwitterionic surfactants suitable for inclusion in a personal care composition along with a glyceryl lauryl azelate according to the present disclosure can be described as surface active agents having a positive and negative charge in the same molecule which molecule is zwitterionic at all pH's. Zwitterionic surfactants can perhaps be best illustrated by the betaines and the sultaines. The zwitterionic compounds generally contain a quaternary ammonium, quaternary phosphonium or a tertiary sulfonium moiety. The cationic atom in the quaternary compound can be part of a heterocyclic ring. In all of these compounds there is at least one aliphatic group, straight chain or branched, containing from about 6 to 20, preferably 8 to 18, carbon atoms and at least one aliphatic substituent containing an anionic water-solubilizing group, e.g., carboxy, sulfonate, sulfate, phosphate or phosphonate.

Examples of amphoteric and zwitterionic surfactants suitable for inclusion in a personal care composition along with a glyceryl lauryl azelate according to the present disclosure include the alkali metal, alkaline earth metal, ammonium or substituted ammonium salts of alkyl amphocarboxyglycinates and alkylamphocarboxypropionates, alkyl amphodipropionates, alkyl monoacetate, alkyl diacetates, alkylamphoglycinates, and alkyl amphopropionates wherein alkyl represents an alkyl group having from 6 to about 20 carbon atoms. Other suitable surfactants include alkyliminomonoacetates, alkyliminidiacetates, alkyliminopropionates, alkyliminidipropionates, and alkylamphopropylsulfonates having between 12 and 18 carbon atoms, alkyl betaines and alkylamidoalkylene betaines and alkyl sultaines and alkylamidoalkylenehydroxy sulfonates.

Anionic surfactants suitable for inclusion in a personal care composition along with a glyceryl lauryl azelate according to the present disclosure are those surfactant compounds which contain a long chain hydrocarbon hydrophobic group in their molecular structure and a hydrophilic group, including salts such as carboxylate, sulfonate, sulfate or phosphate groups. The salts may be sodium, potassium, calcium, magnesium, barium, iron, ammonium and amine salts of such surfactants. Anionic surfactants include the alkali metal, ammonium and alkanol ammonium salts of organic sulfuric reaction products having in their molecular structure an alkyl, or alkaryl group containing from 8 to 22 carbon atoms and a sulfonic or sulfuric acid ester group. Examples of such anionic surfactants include water soluble salts of alkyl benzene sulfonates having between 8 and 22 carbon atoms in the alkyl group, alkyl ether sulfates having between 8 and 22 carbon atoms in the alkyl group and 2 to 9 moles ethylene oxide in the ether group. Other anionic surfactants that can be mentioned include alkylsulfosuccinates, alkyl ethersulfosuccinates, olefin sulfonates, alkyl sarcosinates, alkyl monoglyceride sulfates and ether sulfates, alkyl ether carboxylates, paraffinic sulfonates, mono and di-alkyl phosphate esters and ethoxylated derivatives, acyl methyl taurates, fatty acid soaps, collagen hydrosylate derivatives, sulfoacetates, acyl lactates, aryloxide disulfonates, sulfosucinamides, naphthalene-formaldehyde condensates and the like. Aryl groups generally include one and two rings, alkyl generally includes from 8 to 22 carbon atoms and the ether groups generally range from 1 to 9 moles of ethylene oxide (EO) and/or propylene oxide (PO), preferably EO. Specific anionic surfactants which may be selected include linear alkyl benzene sulfonates such as decylbenzene sulfonate, undecylbenzene sulfonate, dodecylbenzene sulfonate, tridecylbenzene sulfonate, nonylbenzene sulfate and the sodium, potassium, ammonium, triethanol ammonium and isopropyl ammonium salts thereof.

Nonionic surfactants may also be used in combination with the glyceryl lauryl azelate esters of the present disclosure in a personal care or skin-care composition. The nonionic surfactant (s) is not generally critical and may be any of the known nonionic surfactants which are generally selected on the basis of compatibility, effectiveness and economy. Examples of useful nonionic surfactants include condensates of ethylene oxide with a hydrophobic moiety which has an average hydrophilic lipolytic balance (HLB) between about 8 to about 16, and preferably between about 10 and about 12.5. The surfactants include the ethoxylated primary or secondary aliphatic alcohols having from about 8 to about 24 carbon atoms, in either straight or branch chain configuration, with from about 2 to about 40, and preferably between about 2 and about 9 moles of ethylene oxide per mole of alcohol. Other suitable nonionic surfactants include the condensation products of from about 6 to about 12 carbon atoms alkyl phenols with about 3 to about 30, and preferably between about 5 to about 14 moles of ethylene oxide.

Many cationic surfactants are known in the art and almost any cationic surfactant having at least one long chain alkyl group of about 10 to 24 carbon atoms is suitable for optional use as a component in a final formulation which includes a glyceryl lauryl azelate according to the present disclosure.

Other optional ingredients or additives which may be used in combination with glyceryl lauryl azelate esters in formulating personal care compositions according to the present disclosure include pH adjusting chemicals, for example, lower alkanolamines such as monoethanolamine (MEA) and triethanolamine (TEA). Sodium hydroxide solutions may also be utilized as an alkaline pH adjusting agent. The pH adjusting chemicals function to neutralize acidic materials that may be present. Mixtures of more than one pH adjusting chemical can also be utilized.

Phase regulants (well known liquid detergent technology) may also be optionally used in the present disclosure. These can be represented by lower aliphatic alcohols having from 2 to 6 carbon atoms and from 1 to 3 hydroxyl groups, ethers of diethylene glycol and lower aliphatic monoalcohols having from 1 to 4 carbon atoms and the like.

Detergent hydrotropes may also be included. Examples of detergent hydrotropes include salts of alkylarylsulfonates having up to 3 carbon atoms in the alkyl group e.g., sodium, potassium, ammonium, and ethanolamine salts of xylene, toluene, ethylbenzene, cumene, and isopropylbenzenesulfonic acids.

Other optional supplemental additives include de-foamers such as high molecular weight aliphatic acids, especially saturated fatty acids and soaps derived from them, dyes and perfumes; fluorescent agents or optical brighteners; suspension stabilizing agents and soil release promoters such as copolymers of polyethylene terephthalate and polyoxyethylene terephthalate; antioxidants; softening agents and anti-static agents; photo activators and preservatives; polyacids, suds regulators, opacifiers, bacteriacide, and the like. Suds regulants can illustrated by alkylated polysiloxanes and opacifiers can be illustrated by polystyrene; bactericide can be illustrated by butylated hydroxytoluene.

Although not required, an inorganic or organic builder may optionally be added in small amounts to a final composition according to the disclosure. Examples of inorganic builders include water-soluble alkali metal carbonates, bicarbonates, silicates and crystalline and amorphous alumino-silicates. Examples of organic builders include the alkali metal, alkaline metal, ammonium and substituted ammonium polyacetates, carboxylates, polycarboxylates, polyacetyl carboxylates and polyhydroxy sulfonates. One example of a commonly used builder is sodium citrate.

The optional ingredients and optional surfactants can be added to the glyceryl lauryl azelate ester before, during or after formulation of the skin care or personal care formulation. In addition, blends of the glyceryl lauryl azelate ester in combination with these optional ingredients and surfactants can be made directly for sale or for compounding to meet the needs of the user.

The glyceryl lauryl azelate esters of the present disclosure are useful in formulations which contain materials typically used by and known to those skilled in the art as being useful in formulating soap products, skin-care compositions, shampoos and other cleansing products, particularly, but not limited, to personal care cleansers. The words “known to those skilled in the art as being useful in formulating soap products, skin-care compositions, shampoos and other cleansing products” means one or more of the materials selected from the group consisting of fatty acids, alkyl sulfates, ethanolamines, amine oxides, alkali carbonates, water, ethanol, isopropanol, pine oil, sodium chloride, sodium silicate, polymers, alcohol alkoxylates, zeolites, perborate salts, alkali sulfates, enzymes, hydrotropes, dyes, fragrances, preservatives, brighteners, builders, poly-acrylates, essential oils, alkali hydroxides, ether sulfates, alkylphenol ethoxylates, fatty acid amides, alpha olefin sulfonates, paraffin sulfonates, betaines, chelating agents, tallowamine ethoxylates, polyetheramine ethoxylates, ethylene oxide/propylene oxide block copolymers, alcohol ethylene oxide/propylene oxide low foam surfactants, methyl ester sulfonates, alkyl polysaccharides, N-methyl glucamides, alkylated sulfonate di-phenyl oxide, and water soluble alkylbenzene sulfonates or alkyltoluene sulfonates, as the use of such in formulating soaps, detergents, and the cleansing-like products are known in the art.

In one embodiment, the glyceryl lauryl azelate esters of the present disclosure may be present in facial and body cleansing compositions. These cleansing compositions may also comprise a fatty acid soap together with other non-soap surfactants, such as mild synthetic surfactants. Body and facial cleaning compositions may also generally include a moisturizer or emollient and polymeric skin feel and mildness aids. The compositions may further optionally include thickeners (e.g., magnesium aluminum silicate, carbopol), conditioners, water soluble polymers (e.g., carboxymethylcellulose), dyes, hydrotropes brighteners, perfumes and germicides.

In another embodiment, the glyceryl lauryl azelate esters of the present disclosure may be present in a shampoo. The shampoo composition may also comprise one or more other surfactants, optionally a compound considered useful for treating dandruff, such as selenium sulfide, a suspending agent, an amide, nonionic polymer material for aiding in dispersing particles, nonvolatile silicone fluid, and a variety of other nonessential components suitable for rendering the composition more formulatable, such as preservatives, viscosity modifiers, pH adjusting chemicals, perfumes, and dyes.

In still another embodiment, the glyceryl lauryl azelate esters of the present disclosure may be present in a light duty liquid detergent composition. The light duty liquid detergent composition may further include one or more other surfactants, opacifiers (e.g. ethylene glycol di-stearate), thickeners (e.g. guar gum), antimicrobial agents, anti-tarnish agents, heavy metal chelators (e.g. EDTA), perfumes and dyes.

In a further embodiment, the glyceryl lauryl azelate esters of the present disclosure may be present in a heavy duty liquid detergent composition. The heavy duty liquid detergent composition may also include one or more other surfactants, an electrolyte (i.e. water soluble salt), enzymes with or without stabilizers such as calcium ion, boric acid, propylene glycol and/or short chain carboxylic acids, and conventional alkaline detergency builders.

In yet another embodiment, the glyceryl lauryl azelate esters of the disclosure may be present in a conditioner composition that comprises alkylamine compounds.

In a different embodiment, the glyceryl lauryl azelate esters of the present disclosure may be present in a cosmetic composition, such as lipstick, and including lip balms. The cosmetic composition may further include at least one polymer thickening agent, one or more chemical preservatives or water activity depressants to prevent microbial spoilage, a sun-screening agent such as p-aminobenzoic acid, cinnamic acid derivatives, and a vehicle. The vehicle can include any diluent, dispersant or carrier useful in ensuring an even distribution of the composition when applied to skin and may include water, an emollient such as an alcohol or oil, a propellant for example, trichloromethane, carbon dioxide or nitrous oxide, a humectant, and a powder such as chalk, talc, and starch.

Effective anti-microbial amounts of the esters disclosed herein may be orally ingested, using pharmaceutically-acceptable carriers, including in pill and capsule forms.

The examples which now follow shall be considered exemplary of the present disclosure, and not delimitive thereof in any way. All parts are by weight unless otherwise specified.

### EXAMPLE I

### Glyceryl Azelates Concentrate

One mole (137 grams) of dry 2,2-dimethyl-1,3-dioxolane-4-methanol (97%, Alfa Aesar) and two grams of powdered zinc oxide are placed in a three-neck 500 ml round bottom flask equipped with a reflux condenser, addition funnel, heating mantle and magnetic stirrer under gentle agitation. The dropping funnel is charged with about 0.2 moles (44 grams) of the dimethyl ester of azelaic acid, which is added dropwise over the course of about one half hour to the stirred mixture, kept at about 130 degrees centigrade, the methanol liberated being collected in a receiver attached to a side-arm condenser. Following the addition, the temperature of the contents of the flask is maintained for four hours, then cooled to 25 degrees centigrade. The reaction product mixture is next subject to acid hydrolysis in the presence of excess water. The mixture is cooled and the water layer separated. 200 milliliters of water are added slowly and the contents mixed gently to enable glycerine present in the mixture to dissolve in the water, without forming large amounts of emulsion, which water is then decanted. This is repeated three times and finally the resulting product mixture, containing a mixture of the two possible monoazelyl esters of glycerine (glyceryl monoazelates), is taken up in ether and washed gently three times more with 100 ml of water to remove the last traces of glycerine. The product is dried (anhyd. magnesium sulfate) and the ether removed, to yield a glyceryl azelates concentrate from which a composition according to the disclosure may be produced.

### EXAMPLE II

### Shampoo

One gram of the concentrate produced in Example I is mixed with fifty grams of Alberto VO5® shampoo (Alberto-Culver) in a small cup using a spoon to afford an anti-bacterial shampoo useful for treating the hair and scalp.

### EXAMPLE III

### Conditioner

One gram of the concentrate produced in Example I is mixed with fifty grams of TRESemme® Pro-Vitamin B5 & Aloe Remoisturizing conditioner (Alberto-Culver) in a small cup using a spoon to afford an anti-bacterial conditioner useful for treating the hair and scalp.

### EXAMPLE IV

### Skin-Care Composition

One gram of the concentrate produced in Example I is mixed with fifty grams of Fruit of the Earth® Cocoa butter with aloe & vitamin E skin care lotion (Fruit of the Earth, Inc.) in a small cup using a spoon to provide an anti-bacterial skin lotion useful for treating the skin for acne.

### EXAMPLE V

### Hand and Body Soap

One gram of the concentrate produced in Example I is mixed with fifty grams of Softsoap® hand soap (Colgate-Palmolive) in a small cup using a spoon to provide an anti-microbial soap composition.

### EXAMPLE VI

### Glyceryl Lauryl Azelates

One hundred grams of 1-glyceryl monoazelate is taken up in 500 ml of toluene and heated to 80 degrees centigrade. A solution of 0.6 moles (130 grams) of lauryl chloride dissolved in 200 ml of toluene is slowly added over the course of two hours, with stirring, maintaining the temperature at 80 degrees centigrade for four hours. The reaction mixture is cooled to room temperature, and mixed with an equal volume of water. After six hours, the water layer is discarded and the residue is treated slowly with one liter of 10% sodium bicarbonate solution, after which the water layer is again discarded. The residue is washed three times with water, then acidified with 100 ml of 37% aqueous HCl, washed again with water, dried and the toluene and residual HCl removed under reduced pressure to yield a mixture containing compounds conforming to the structures (I), (II), in which R1, R2, and R3 are hydrogen. This material may be readily purified by molecular distillation or column chrmoatography and is suitable for use in the place of the concentrate of Example I described in the compositions provided according to examples II, III, IV, and V, and for providing other formulations described herein.

### EXAMPLE VII

### Glyceryl Azelates

Example I is repeated, except that glycerine is employed in the place of the glyceryl ketal of acetone previously used as reactant, to afford a crude product mixture comprising glyceryl monoazelate having the azelaic moiety present on both the 1-position and 2-position of the glycerine molecule. The crude product is purified by molecular distillation to separate these compounds. The material having an azelaic group in the 1-position of the glycerine may be treated with an equimolar amount of acid chloride of lauric acid (lauryl chloride) per the procedure of Example VI to yield a compounds corresponding to formula (I) and (II), and the material having an azelaic group in the 2-position of the glycerine may be treated with an appropriate molar amount of acid chloride of lauric acid (lauryl chloride) per the procedure of Example VI to yield a compounds corresponding to formula (III) and (IV). These materials may be readily worked up and are suitable for use in the place of the concentrate of Example I described in the compositions provided according to examples II, III, IV, and V, and for providing other formulations described herein. These materials may be further purified before use by molecular distillation.

### EXAMPLE VIII

### Glyceryl Lauryl Azelates

27.6 grams azelaic acid (0.146 mol) was dissolved in 125 ml of dry acetone in a first beaker on a hot plate at 50 degrees centigrade. Into a 400 ml beaker was charged 129.3 grams of glyceryl monolaurate and 250 ml of acetone, the contents of the beaker were heated to 50 degrees centigrade and stirred until the glyceryl monolaurate had dissolved. Then, 1.0 ml of concentrated (98%) sulfuric acid was added dropwise, with stirring. Immediately after the sulfuric acid had been added, the contents of the first beaker containing azelaic acid dissolved in acetone were poured into the 400 ml beaker, and the contents of the combined contents of the 400 ml beaker were heated at 52 degrees centigrade, with moderate stirring using a stir bar, for 10 hours. After 10 hours, the heat was turned off, the mixture permitted to cool. Into a 500 ml separatory funnel was placed 300 ml of de-ionized water, and half the beaker's contents were poured into the separatory funnel. An oily layer separated, the lower water layer was removed, and the oil washed twice subsequently with two further a liquots of water. The oily layer was collected in a separate beaker, and the procedure repeated for the remaining contents of the reaction beaker, the second crop of oily layer being combined with the first. These combined oil layers were then placed in a 400 ml beaker and then placed on the hot plate and gently heated to 50 degrees centigrade with stirring while passing a gentle stream of air over the beaker's contents, to remove excess acetone, until the odor of acetone was only slightly detectable by sense of smell. The oily layer was now clear and was permitted to cool to room temperature at which time it solidified into a pure white solid, having superb emollient properties. This solid comprises the material having structure (II), a small amount of material having structure (III), with R1, R2 both being hydrogen in each case, and may be purified further if desired by molecular distillation or column chromatography. However, the residual acidity level from the sulfuric acid is very low from the water washings. These materials are suitable for use in the place of the concentrate of Example I in the compositions provided according to examples II, III, IV, and V, inter alia. These materials having structures (II) and (III) with R1, R2 both being hydrogen are believed to be superior in their effectiveness against acne and papulopustular rosacea when applied topically to human skin at concentration levels of 5%, and as low as 1%, as compared with conventional treatments comprising 20% azaleic acid.

### EXAMPLE IX

### Glyceryl Lauryl Azelates

One mole of glyceryl monolaurate (274.45 grams) is placed in a 500 ml round bottom flask equipped with a heating mantle and magnetic stirring bar, and heated to 120 degrees centigrade, to the liquid state. One gram of 85% phosphoric acid catalyst is then added to the stirred molten monolaurate ester, followed by addition of 0.2 grams of hydroquinone (alternatively p-methoxyphenol). One mole of azelaic acid (188.25 grams) is slowly added to the molten glyceryl monolaurate/catalyst mixture with moderate mixing and the temperature raised to 140 degrees centigrade. After the addition of the azelaic acid, a Dean-Stark trap is affixed to the flask and the temperature maintained in the range 135-145 degrees centigrade for one hour or until about one mole of water is collected. The heat is turned off and the material transferred to a 1000 ml beaker. After cooling to ambient temperature, this solid comprises the material having structure (II), a small amount of material having structure (III), with R1, R2 both being hydrogen in each case. The reaction product may be washed with water to remove most of the phosphoric acid. This material may be purified further if desired by washings and subsequent molecular distillation or column chromatography. The material is suitable for use in the place of the concentrate of Example I in the compositions provided according to examples II, III, IV, and V, and for providing other formulations described herein.

### EXAMPLE X

### Zinc Glyceryl Lauryl Azelate

The product from Example VIII or Example IX is heated to melting, and treated with powdered hydrozincite corresponding to Zn₅(CO₃)₂(OH)₆ in the appropriate stoichiometric quantity, which is preferably one zinc atom for every molecule of GLA acid, to afford zinc salts of materials corresponding to structures (VI) and (VII) in which R2 is hydrogen, M=Zn, n=2 and p=2. These zinc salts, termed “Zinc GLA” herein, may be combined with dermatologically acceptable carriers for application to mammalian skin. These salts may be washed and dried and isolated, and formulations containing same in any amounts between 0.01% and 50% by weight based on the total weight of the formulation are undoubtedly very powerful inhibitors of 5-alpha reductase, owing to the release of both zinc and azelaic acid following dermal penetration and cleavage by dermal esterase.

### EXAMPLE XI

### Stick Deodorant Formulation

| Component | % w/w |
|---|---|
| GLA according to structure (II) having R1 and R2 both | 5 |
| being hydrogen | |
| Stearic acid | 8 |
| Ethanol | 72.6 |
| Propylene Glycol | 9 |
| Isopropyl palmitate | 3 |
| Sodium hydroxide (50%) | 2.4 |

The components are mixed at 50 degrees C. and permitted to cool.

### EXAMPLE XII

### Stick Deodorant Formulation

| Component | % w/w |
|---|---|
| Cyclomethicone | 45 |
| Stearyl Alcohol | 19 |
| Dimethicone | 5 |
| GLA according to structure (II) having R1 and R2 both | 22 |
| being hydrogen | |
| Talc | 4 |
| Mineral Oil | 2 |
| Hydrogenated castor oil | 3 |
| Fragrance | q.s. |

The components are mixed at 55 degrees C. and permitted to cool.

### EXAMPLE XIII

### Lip Balm

| Component | % w/w |
|---|---|
| Hydrogenated vegetable oil and/or candelia wax | 66.2 |
| GLA according to structure (II) having R1 and R2 both | 20 |
| being hydrogen | |
| Shea butter extract | 2 |
| Beeswax | 7.8 |
| Carnuba wax | 3.8 |
| Flavor | 0.2 |

The components are combined and mixed at 65 degrees C and permitted to cool.

### EXAMPLE XIV

### Lip Balm

| Component | % w/w |
|---|---|
| Dimethicone NF | 2 |
| Cetyl alcohol NF | 6 |
| Silicone Dioxide NF | 0.2 |
| Cyclomethicone | 7 |
| Colorant | 0.1 |
| GLA according to structure (II) having R1 and R2 both | 3 |
| being hydrogen | |
| Isocetyl stearate | 25.4 |
| Methylparaben NF | 0.1 |
| Microcrystalline Wax | 27 |
| Propylparaben NF | 0.1 |
| Vitamin A Palmitate USP | 0.1 |
| Vitamin E acetate USP | 1 |
| Regent Petrolatum USP | 27 |
| Aloe Vera Oil - filtered | 1 |

### EXAMPLE XV

### Night Cream

| Component | parts |
|---|---|
| Kahl Cream Base 6065 (Lambent Technologies Inc.) | 6 |
| Cetearyl Isononanoate | 8 |
| Jojoba oil | 3 |
| Myristyl Myristate | 4 |
| GLA according to structure (II) having R1 and R2 both | 5 |
| being hydrogen | |
| Siliconized Beeswax 6423 WD (Kahl, Lambent) | 0.5 |
| Water | 68.3 |
| Glycerol | 3 |
| Ammonium acryloyldimethyltaurate/VP copolymer | 0.25 |
| Preservative | 0.10 |

Heat waxes and oils to 75° C. Premix water phase and heat to 75° C. Add water phase to wax/oil phase. Stir and cool to 40° C. Add preservative and fill containers.

### EXAMPLE XVI

### Lipstick

| Component | parts |
|---|---|
| Kahl Lipstick Base 6065 (Lambent Technologies Inc.) | 78 |
| GLA according to structure (II) having R1 and R2 both | 1 |
| being hydrogen | |
| Cosmetic grade castor oil | 4.9 |
| Sun COD 8003 | 2 |
| Sun COD 8006 brown (50% ig) | 5 |
| Sun Shine Glitter Golden C84-3412 | 1 |
| Sun Pearl Glittering White C80-1501 | 0.5 |
| Sun Shine Super Bronze C84-6282 | 1 |
| Sun Shine Glitter Bronze C84-6480 | 4 |
| Sun Pearl Golg C84-6111 | 2.5 |
| Paraben preservative | 0.3 |

Mix castor oil, GLA, pigments and preservative. Homogenize. Melt Lipstick Base at 90° C. Combine with homogenized castor oil, GLA, pigments and preservative mixture. Homogenize. Adjust temperature to 80° C. Pour into mold and cool to 45° C. with water. Cool again to 8° C., demold and pack.

### EXAMPLE XVII

### Hand and Body Cream

| | |
|---|---|
| Component | parts |

Part A

| | |
|---|---|
| Water | 79 |
| Glycerine | 3 |

Part B

| | |
|---|---|
| Stearyl alcohol | 1.5 |
| Cetyl alcohol | 1.8 |
| LUMULSE ® CS-20 (Cetaeareth-20) (LUMULSE ® is a | 1.5 |
| trademark of Lambent technologies Inc.) | |
| GLA according to structure (II) having R1 and R2 both | 3 |
| being hydrogen | |
| LUMULSE ® 602-S (PEG-150 distearate | 1 |

Part C

| | |
|---|---|
| Sodium hydroxide/citric acid | q.s. |
| Preservative | q.s. |
| Fragrance | q.s. |

Heat A to 70° C. and add B components to A with good mixing. Remove heat and homogenize. Transfer to low-shear sweep blade and continue mixing with low agitation while cooling. At 35° C., adjust pH to 6.8 with NaOH or H3Cit. Add remaining components of C in sequential order until well blended. Maintain mixing until material reaches ambient temperature. Pack.

### EXAMPLE XVIII

### Moisturizing Cream

| | |
|---|---|
| Component | parts |

Part A

| | |
|---|---|
| Water | 79 |
| Glycerine | 3 |

Part B

| | |
|---|---|
| Stearyl alcohol | 1.5 |
| Cetyl alcohol | 1.8 |
| LUMULSE ® CS-20 (Cetaeareth-20) (LUMULSE ® is a | 1.5 |
| trademark of Lambent technologies Inc.) | |
| GLA according to structure (II) having R1 and R2 both | 1 |
| being hydrogen | |
| Lambent F-350 | 0.5 |
| Erucical EG-20 (Lambent) | 2 |
| Kahl 6423 WD (siliconized beeswax) | 1 |
| LUMULSE ® 602-S K | 1.5 |
| Lanolin or synthetic substitute | 0.1 |
| LUMULSE ® CC-33K | 2 |
| LUMULSE ® L-4 | 0.5 |
| LAMCHEM ® PE-130 K | 0.5 |

Part C

| | |
|---|---|
| Sodium hydroxide (2% aqueous soln.) | 0.16 |
| Preservative | q.s. |
| Fragrance | q.s. |

Heat A components together to 70° C. Combine B components with each other an heat to 78° C. Combine B and A with good mixing. Remove heat and homogenize. Transfer to low-shear sweep blade and continue mixing with low agitation while cooling. At 35° C., add each component of C in sequential order and mix until well blended. Adjust pH to 6.5-7 with NaOH or H3Cit. Maintain mixing until material reaches ambient temperature. Pack.

### EXAMPLE XIX

### Moisturizing Cream

| | |
|---|---|
| Component | parts |

Part A

| | |
|---|---|
| Water | 79 |
| Glycerine | 3 |

Part B

| | |
|---|---|
| Stearyl alcohol | 1.5 |
| Cetyl alcohol | 1.8 |
| LUMULSE ® CS-20 (Cetaeareth-20) (LUMULSE ® is a | 1.5 |
| trademark of Lambent technologies Inc.) | |
| GLA according to structure (II) having R1 and R2 both | 20 |
| being hydrogen | |
| Lambent F-350 | 0.5 |
| Erucical EG-20 (Lambent) | 2 |
| Kahl 6423 WD (siliconized beeswax) | 1 |
| LUMULSE ® 602-S K | 1.5 |
| Lanolin or synthetic substitute | 0.1 |
| LUMULSE ® CC-33K | 2 |
| LUMULSE ® L-4 | 0.5 |
| LAMCHEM ® PE-130 K | 0.5 |

Part C

| | |
|---|---|
| Sodium hydroxide (2% aqueous soln.) | 0.16 |
| Preservative | q.s. |
| Fragrance | q.s. |

Heat A components together to 70° C. Combine B components with each other an heat to 78° C. Combine B and A with good mixing. Remove heat and homogenize. Transfer to low-shear sweep blade and continue mixing with low agitation while cooling. At 35° C., add each component of C in sequential order and mix until well blended. Adjust pH to 6.5-7 with NaOH or H3Cit. Maintain mixing until material reaches ambient temperature. Pack.

### EXAMPLE XX

### Moisturizing Cream

| | |
|---|---|
| Component | parts |

Part A

| | |
|---|---|
| Water | 79 |
| Glycerine | 3 |

Part B

| | |
|---|---|
| Stearyl alcohol | 1.5 |
| Cetyl alcohol | 1.8 |
| LUMULSE ® CS-20 (Cetaeareth-20) (LUMULSE ® is a | 1.5 |
| trademark of Lambent technologies Inc.) | |
| Zinc salt of GLA according to structure (II), R2 | 20 |
| being hydrogen | |
| Lambent F-350 | 0.5 |
| Erucical EG-20 (Lambent) | 2 |
| Kahl 6423 WD (siliconized beeswax) | 1 |
| LUMULSE ® 602-S K | 1.5 |
| Lanolin or synthetic substitute | 0.1 |
| LUMULSE ® CC-33K | 2 |
| LUMULSE ® L-4 | 0.5 |
| LAMCHEM ® PE-130 K | 0.5 |

Part C

| | |
|---|---|
| Sodium hydroxide (2% aqueous soln.) | 0.16 |
| Preservative | q.s. |
| Fragrance | q.s. |

Heat A components together to 70° C. Combine B components with each other an heat to 78° C. Combine B and A with good mixing. Remove heat and homogenize. Transfer to low-shear sweep blade and continue mixing with low agitation while cooling. At 35° C., add each component of C in sequential order and mix until well blended. Adjust pH to 6.5-7 with NaOH or H3Cit. Maintain mixing until material reaches ambient temperature. Pack.

### EXAMPLE XXI

### Shampoo

| | |
|---|---|
| Component | parts |

Part A

| | |
|---|---|
| Deionized water | q.s. |
| Sodium lauryl sulfate | 6.8 |
| Sodium laureth sulfate | 18.5 |
| Tetrasodium EDTA | 0.2 |

Part B

| | |
|---|---|
| LUMULSE ® 602-S (PEG di-stearate) | 1.5 |

Part C

| | |
|---|---|
| LUMISORB PSML-80 (PEG-80 sorbitan laurate) | 11.2 |
| GLA according to structure (II) having R1 and R2 both | 1 |
| being hydrogen | |
| Cocamidopropyl Betaine | 12.8 |
| LUMULSE ® L-4 | 1 |

Part D

| | |
|---|---|
| Citric acid | q.s. |
| Glycerine | 1.5 |
| Preservative | q.s. |
| Fragrance | q.s. |

Heat A to 60° C. Add B while mixing and maintaining heat until completely dissolved. Cool to 35° C. Adjust pH to about 6.5 with citric acid. Add remaining ingredients and mix until homogeneous.

### EXAMPLE XXII

### Shampoo

| | |
|---|---|
| Component | parts |

Part A

| | |
|---|---|
| Deionized water | q.s. |
| Sodium lauryl sulfate | 6.8 |
| Sodium laureth sulfate | 18.5 |
| Tetrasodium EDTA | 0.2 |

Part B

| | |
|---|---|
| LUMULSE ® 602-S (PEG di-stearate) | 1.5 |

Part C

| | |
|---|---|
| LUMISORB PSML-80 (PEG-80 sorbitan laurate) | 11.2 |
| GLA according to structure (II) having R1 and R2 both | 25 |
| being hydrogen | |
| Cocamidopropyl Betaine | 12.8 |
| LUMULSE ® L-4 | 1 |

Part D

| | |
|---|---|
| Citric acid | q.s. |
| Glycerine | 1.5 |
| Preservative | q.s. |
| Fragrance | q.s. |

Heat A to 60° C. Add B while mixing and maintaining heat until completely dissolved. Cool to 35° C. Adjust pH to about 6.5 with citric acid. Add remaining ingredients and mix until homogeneous.

### EXAMPLE XXIII

### Shampoo

| | |
|---|---|
| Component | parts |

Part A

| | |
|---|---|
| Deionized water | q.s. |
| Sodium lauryl sulfate | 6.8 |
| Sodium laureth sulfate | 18.5 |
| Tetrasodium EDTA | 0.2 |

Part B

| | |
|---|---|
| LUMULSE ® 602-S (PEG di-stearate) | 1.5 |

Part C

| | |
|---|---|
| LUMISORB PSML-80 (PEG-80 sorbitan laurate) | 11.2 |
| Zinc salt of GLA according to structure (II), R2 | 20 |
| being hydrogen | |
| Cocamidopropyl Betaine | 12.8 |
| LUMULSE ® L-4 | 1 |

Part D

| | |
|---|---|
| Citric acid | q.s. |
| Glycerine | 1.5 |
| Preservative | q.s. |
| Fragrance | q.s. |

Heat A to 60° C. Add B while mixing and maintaining heat until completely dissolved. Cool to 35° C. Adjust pH to about 6.5 with citric acid. Add remaining ingredients and mix until homogeneous.

Consideration must be given to the fact that although this disclosure has been put forth in relation to certain preferred embodiments, obvious equivalent modifications and alterations thereof will become apparent to one of ordinary skill in this art upon reading and understanding this specification and the claims appended hereto. This includes subject matter defined by any combination of any one of the various claims appended hereto with any one or more of the remaining claims, including the incorporation of the features and/or limitations of any dependent claim, singly or in combination with features and/or limitations of any one or more of the other dependent claims, with features and/or limitations of any one or more of the independent claims, with the remaining dependent claims in their original text being read and applied to any independent claims so modified. This also includes combination of the features and/or limitations of one or more of the independent claims with features and/or limitations of another independent claims to arrive at a modified independent claim, with the remaining dependent claims in their original text being read and applied to any independent claim so modified. Accordingly, the presently disclosed disclosure is intended to cover all such modifications and alterations, and is limited only by the scope of the claims which follow.

What is claimed is:

1) A composition of matter useful for topical application to mammalian skin, which comprises at least one ester compound having a structure selected from the group consisting of: 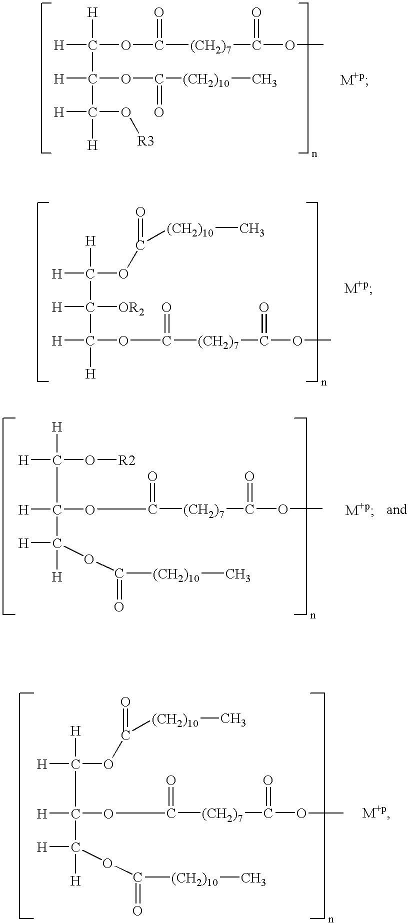
wherein M, in each occurrence, is selected from the group consisting of: hydrogen, a metal cation, a substituted or unsubstituted ammonium cation, any C1-C18 hydrocarbyl group, and a glyceryl group having the structure: 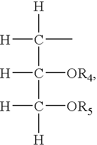
in which R4 and R5 are in each occurrence independently selected from the group consisting of: hydrogen and any C1-C18 hydrocarbyl group;
R2 and R3 are each, in each occurrence, independently selected from the group consisting of: hydrogen, and any C1-C18 hydrocarbyl group, and
wherein p is the valency of the species M, and n equals p.

2) A composition according to claim 1 wherein the amount of said at least one ester compound in said composition is any amount between about 0.01% by weight to about 90% by weight based on the total weight of said composition.

3) A composition according to claim 1 wherein said at least one ester compound is a compound wherein M comprises hydrogen.

4) A composition according to claim 1 wherein said at least one ester compound is a compound wherein M and R2 both comprise hydrogen.

5) A composition according to claim 1 wherein said at least one ester compound is a compound wherein M and R3 both comprise hydrogen.

6) A composition according to claim 1 in which M is a metal cation selected from the group consisting of: lithium, sodium, potassium, calcium, magnesium, strontium, aluminum, zirconium, silver, copper, and zinc.

7) A composition according to claim 1 in which M is zinc.

8) A composition according to claim 1 in which M is sodium.

9) A composition according to claim 1 in which M is a cation selected from the group consisting of: mono-alkylammonium cations, di-alkylammonium cations, tri-alkylammonium cations, and quaternaryalkylammonium cations.

10) A composition according to claim 1 wherein M is selected from the group consisting of: methyl and ethyl, and R2 comprises hydrogen.

11) A composition according to claim 1 wherein M is selected from the group consisting of: methyl and ethyl, and R3 comprises hydrogen.

12) A composition according to claim 1, further comprising at least one material known to those skilled in the art as being useful in formulating soap products, skin-care compositions, shampoos and other cleansing products.

13) A composition according to claim 1 wherein said composition is a composition selected from the group consisting of: skin creams; shampoos; lip balms; hair conditioners; hand soaps; body soaps; stick deodorants; lipsticks; and moisturizing creams, and wherein said at least one ester is present in any amount between about 0.1% and 50% by weight, based on the total weight of said composition.

14) A method for treating mammalian skin, said method comprising topically applying to skin areas to be treated a composition which comprises an ester compound having a structure selected from the group consisting of: 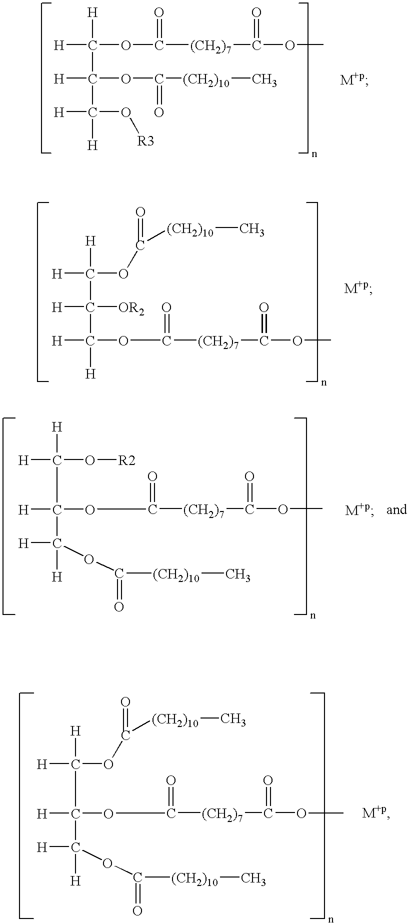
wherein M, in each occurrence, is selected from the group consisting of: hydrogen, a metal cation, a substituted or unsubstituted ammonium ion, any C1-C18 hydrocarbyl group, and a glyceryl group having the structure: 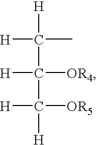
in which R4 and R5 are in each occurrence independently selected from the group consisting of: hydrogen and any C1-C18 hydrocarbyl group;
R2 and R3 are each, in each occurrence, independently selected from the group consisting of: hydrogen, and any C1-C18 hydrocarbyl group, and
wherein p is the valency of the species M, and n equals p.

15) A process according to claim 14 wherein said mammalian skin is selected from the group consisting of: equestrian skin, canine skin, feline skin, and human skin.

16) A process according to claim 14 wherein M and R2 both comprise hydrogen.

17) A process according to claim 14 wherein M is zinc, p and n each equal two, and wherein R2 comprises hydrogen.

18) A process according to claim 14 wherein said composition comprises between about 0.1% and 50% by weight based on the total weight of said composition, of at least one of said ester compounds, and wherein said composition is applied to acne vulgaris on said skin.

19) A process according to claim 14 wherein said composition comprises between about 0.1% and 50% by weight based on the total weight of said composition, of at least one of said ester compounds, and wherein said composition is applied to papulopustular rosacea on said skin.

20) A process according to claim 14 wherein said composition comprises between about 0.1% and 50% by weight based on the total weight of said composition, of at least one of said ester compounds wherein M is zinc, and wherein said composition is applied to an area on said skin where dormant hair follicles are present.
